(19) United States
(12) Patent Application Publication
(10) Pub. No.: US 2014/0255351 A1
(43) Pub. Date: Sep. 11, 2014

(54) Composition Comprising Anaerobically Cultivated Human Intestinal Microbiota

(71) Applicant: ACHIM Biotherapeutics AB, Vällingby (SE)

(72) Inventors: Arnold Berstad, Oslo (NO); Tore Midtvedt, Solna (SE); Elisabeth Norin, Vällingby (SE); Peter Benno, Lidingö (SE); Atti-La Dahlgren, Neuchatel (CH)

(73) Assignee: ACHIM Biotherapeutics AB, Vällingby (SE)

(21) Appl. No.: 14/351,076

(22) PCT Filed: Oct. 11, 2012
(86) PCT No.: PCT/EP2012/070177

§ 371 Date: Apr. 10, 2014

(30) Foreign Application Priority Data
Oct. 11, 2011 (SE) 1150940-3
Jan. 11, 2012 (SE) 1250011-2

(51) Int. Cl. A61K 35/74

(52) U.S. Cl. CPC A61K 35/74
USPC 424/93.3; 435/252.3

(57) ABSTRACT

The invention relates to an anaerobic micro-ecological system, a composition comprising anaerobically cultivated human intestinal microbiota for the treatment or prophylaxis of diseases, and wherein the microbiota in the composition have not been exposed to antibiotics used after 1995. The present invention further relates to the use of the composition for the prevention and treatment of disease and a method for preparing the composition.

### FIELD OF THE INVENTION

The present invention relates to an anaerobic micro-ecological system comprising anaerobically cultivated human intestinal microbiota. The present invention also relates to a composition of anaerobically cultivated microbiota that constitutes a functional seeding culture for re-establishing normality of a disturbed human microbiome and gastrointestinal functions.

The present invention further relates to the use of the composition for the prevention and treatment of disease and a method for preparing the composition.

### TECHNICAL BACKGROUND

The gastrointestinal microbiota consists of several complex micro-ecological systems, where interactions in-between the individual microbial strains and between the microbiota and the host takes place. The interactions with the host occur through direct contact with the mucosal wall and indirectly e.g. through metabolites and signalling substances.

This intestinal ecological balance can be disturbed by external and internal physical, chemical and biological factors or agents, the consequence of which can be local e.g. disturbed metabolism, gastrointestinal motility alteration, altered functions/crosstalks, andor lead to systemic effects involving other organ systems.

Clostridium difficile associated diarrhoea CDAD was first described around ⅓ of a century ago. However, in spite of thousands of publications in Pub Med of the pathogenesis, epidemiology, clinical diagnosis and various therapeutic approaches CDAD continues to persist as a costly, leading cause of infectious health-care-associated gastrointestinal illness and the problems are assumed to increase worldwide. It was recently stated that today we experienced a tenfold increase of CDAD in Europe, USA and Canada. Increasing therapeutic failures of metronidazole and vancomycin treatments are reported and new antibiotics, as fidaxomicin, oritavancin, nitazoxanide, REP3123, and NVB303 are on their way to the market. However, it has to be recognized that even before these drugs have been used, a certain number of Clostridium difficile strains are expressing a reduced sensitivity to these new drugs. Additionally, whatever their clinical efficacy may turn out to be, they will certainly be expensive in use.

It has been stated that some patients continue to manifest relapsing diarrhoea after completed treatment with novel drugs, and therefore other strategies have been focused on e.g. faecal transplantation from a close relative or a synthetic composition comprising a mixture of different bacteria.

WO 02/07741 describes a synthetic composition comprising a preparation of a predetermined flora for the treatment of gastrointestinal disorders. WO 2011/033310 describes encapsulated dosage form comprising flora extracted from faeces for use in the treatment of gastrointestinal disorders.

Faecal transplantation is a known method, but not without risk. Until now faecal material from donors of close relatives has been used. A main problem with this procedure is the risk of transmission of genes resistant to certain antibiotics. The transfer of such genes may have serious consequences for the patient when antibiotic treatment is needed in the future. Another main problem with this therapy is the risk of transmission of potentially contagious agents present in the donor's faeces, which argue for careful screening of the faeces to be given. This screening is time-consuming and expensive. Further this type of treatment is perceived by many as highly unesthetical.

Previous publications mentioning faecal transplantation:
Johan S. Bakken Fecal bacteriotherapy for recurrent Clostridium difficile infection Anaerobe 15 (2009) 285-289.
C Jorup-Rönström, A Håkansson, A-K Person,T Midtvedt, E Norin. Feceskultur framgångsrik terapi vid Clostridium difficile-diarre. Läkartidningen nr 46 2006 volym 103, 3603-3605.
A. Gustafsson, A. Berstad, S. Lund-Tønnesen, T. Midtvedt, E. Norin. The Effect of Faecal Enema on Five Microflora-Associated Characteristics in Patients with Antibiotic-Associated Diarrhoea. Scand J Gastroenterol 1999;34:580-586.
A. Gustaysson, S. Lund-Tønnesen, A. Berstad, T. Midtvedt & E. Norin Faecal Short-Chain Fatty Acids in Patients with Antibiotic-Associated Diarrhoea, before and after Faecal Enema Treatment. Scand J Gastroenterol 1998;33:721-727.

### SUMMARY OF THE INVENTION

The present invention relates to an anaerobically cultivated micro-ecological system comprising anaerobically cultivated human intestinal microbiota.

The present invention further relates to a composition comprising said microbiota for pharmaceutical treatment and prophylaxis of diseases and a method to prepare such a composition. It further comprises the administration of a therapeutic or prophylactic amount of the invention to a human.

The present invention also relates to a composition comprising anaerobically cultivated intestinal microbiota that constitutes a functional seeding culture for re-establishing normality of a disturbed human microbiota and gastrointestinal functions and the use of such composition for the prevention and treatment of disease.

This invention also relates to an anaerobically cultivated human intestinal microbiota for the pharmaceutical treatment of gastrointestinal andor systemic/metabolic disorders that has its origin in a disturbed or dysfunctional gastrointestinal ecosystem.

The invention also relates to the use of such an anaerobically cultivated human microbiota for pharmaceutical treatment or prevention of disease. The composition may also contain other microbial factors (e.g. bacteriophages) or signal substances active against diseases.

### DETAILED DESCRIPTION OF THE INVENTION

A first aspect of the present invention is an anaerobic micro-ecological system comprising anaerobically cultivated human intestinal microbiota.

Another aspect of the present invention is a composition comprising anaerobically cultivated human intestinal microbiota.

In the present application the term “microbiota” is used for the microbial flora, i.e. the microorganisms that typically inhabit the gastrointestinal system.

The composition according to the present invention constitutes a functional cultivate for re-establishing normality of a disturbed human microbiota and gastrointestinal functions. This composition has surprisingly shown to be useful as in the treatment or prophylaxis of disease.

The composition according to the present invention interacts in a synergistic way with the damaged intestinal microbiota and re-establish its physiological and metabolic functions. The invention may also be used as a prophylactic to avoid the disturbance of the intestinal microbiota in the first place.

According to one embodiment the composition contains anaerobic bacteria of at least three of the following four Phyla: Bacterioidetes, Firmicutes, Proteobacteria and Actinobacteria.

According to another embodiment the composition is free from the following microbes: hepatitis viruses A, B and C, cytomegalo virus, Epstein-Barr virus, human immunodeficiency virus (HIV), Calici- and Rotavirus, Salmonella, Shigella, Campylobacter, Yersinia, and protozoan cysts. Preferably it is also free from material selected from Extended spectrum- and Metallo-beta-lactamases, metabolites of pharmaceutical substances, and xenobiotics.

Preferably the microbiota in the composition according to the present invention have not been exposed to antibiotics taken into clinical use after 1995.

This invention comprises an anaerobically cultivated human intestinal microbiota for the pharmaceutical treatment of gastrointestinal andor systemicmetabolic disorders that has its origin in a dysfuntional gastrointestinal ecosystem.

An advantage with the composition according to the present invention is that it consists of functional micro-ecological system that is stable and homogenous during 15 years of cultivation. No other bacterial strains enter the system during cultivation. The microbial strains in the micro-ecological system interact and communicate with each other. A micro-ecological system is homogenous.

The functional ecologic system according to the present invention has the ability to re-establish andor stabilise intestinal microbiota andor normal intestinal functions.

Another aspect of the present invention is a composition comprising a micro-ecological system comprising anaerobically cultivated human intestinal microbiota for the treatment or prophylaxis of diseases.

Preferably, the bacterial content of the composition is more than 10⁶ per ml medium, more preferably more than 10⁹ per ml medium.

In one embodiment of the present invention the composition comprises an anaerobic micro-ecological system obtained from faeces from a single donor. Cultivation of faeces from a healthy single donor is necessary for obtaining a functional micro-ecological system that is stable over time.

Preferably, the donor is a healthy individual with a well-functional gastrointestinal system.

Preferably, the faeces is obtained from a single stool sample. Also preferably, the faeces were obtained prior to 1995.

In a preferred embodiment of the present invention, the composition is kept under strict anaerobic conditions until the final administration. Also, preferably the composition is kept under anaerobic conditions during the final administration.

Another aspect of the present invention is a method for preparing a composition comprising an anaerobic micro-ecological system comprising anaerobically cultivated human intestinal microbiota.

The composition according to the present invention has been cultivated under strict anaerobic conditions. By anaerobic cultivation the proliferation of the anaerobic bacteria is promoted, and further proliferation of aerobic bacteria is suppressed.

In a preferred embodiment of the present invention, the composition is prepared by cultivating a stool sample obtained from a healthy individual. Preferably, the healthy individual has normal functional values of coprostanol, urobilins, mucin, fecal tryptic activity, short chain fatty acids and beta-aspartylglycine. Also preferably, the sample should be obtained prior to 1995. More preferably the stool sample is investigated to be free from hepatitis viruses A, B and C, cytomegalovirus, Epstein-Barr virus, human immunodeficiency virus (HIV), Calici- and Rotavirus. Furthermore, the stool sample is screened to be free from Salmonella, Shigella, Campylobacter, Yersinia, Clostridium difficile and the presence of protozoan cysts.

Preferably the cultivating medium used in the method according to the invention promotes the proliferation of the anaerobic bacteria of at least three of the following four Phyla: Bacterioidetes, Firmicutes, Proteobacteria and Actinobacteria.

In one embodiment the cultivation medium is a yeast based medium, preferably a bacteriological peptone yeast based medium. The cultivation medium comprises added cholesterol, preferably cholesterol of animal origin. Even more preferably the medium comprises added freeze dried hen yolk, preferably in an amount of from 0.5 to 5% more preferably 1.25% wv (weight per volume).

Preferably, the cultivation contains an anaerobic indicator, preferably resazurin.

The cultivating medium used in the method according to the invention promotes the proliferation of the anaerobic bacteria of at least three of the following four

Phyla: Bacterioidetes, Firmicutes, Proteobacteria and Actinobacteria in one and the same sample. Further, the preferred cultivating conditions suppresses the proliferation of aerobic bacteria.

In a preferred embodiment the composition is re-cultivated under strict anaerobic conditions, preferably under nitrogen flow every second week.

Preferably, the faecal sample is anaerobically cultivated for at least 15 years.

Cultivating a faecal sample over time according to the method of the present invention promotes the proliferation of the desired bacteria, while aerobic bacteria are suppressed.

An advantage with the method according to the present invention is that it enables proliferation of all desired bacteria in one and the same sample. Thus, no mixing of different cultures is necessary. This has the advantage that the obtained composition comprises a micro-ecological system that has a stabilizing effect on the intestinal microbiota and is able to re-establish the communication between the host and the intestine. Further, by anaerobically cultivating microbiota in one sample, according to the claimed invention, the same micro-ecological system is reproduced in each composition with retained physiological andor pharmaceutical effect over a long time, such as over at least 15 years.

Further, the micro-ecological system according to the present invention is cultivated under strict anaerobic conditions.

The human intestinal flora consists of several hundreds of different microbes, of which the vast majority are strictly anaerobic.

In one embodiment of the invention the following bacterial species together comprise more than 1% preferably more than 20%, and more preferably more than 50% of the total bacterial content of the composition;

### Bacteria;“Actinobacteria”;Actinobacteria;Coriobacteridae;Coriobacteriales;“Coriobacterineae”;Coriobacteriaceae;Collinsella,

### Bacteria;“Actinobacteria”;Actinobacteria;Coriobacteridae;Coriobacteriales;“Coriobacterineae”;Coriobacteriaceae;Eggerthella,

### Bacteria;“Actinobacteria”;Actinobacteria;Coriobacteridae;Coriobacteriales;“Coriobacterineae”;Coriobacteriaceae;Olsenella,

### Bacteria;“Bacteroidetes”;“Bacteroidia”;“Bacteroidales”;“Porphyromonadaceae”; Parabacteroides,

Bacteria;“Bacteroidetes”;“Bacteroidia”;“Bacteroidales”;“Prevotel laceae”; Prevotella,

### Bacteria;“Bacteroidetes”;“Bacteroidia”;“Bacteroidales”;“Rikenellaceae”;Alistipes,

### Bacteria;“Bacteroidetes”;“Bacteroidia”;“Bacteroidales”; Bacteroidaceae; Bacteroides,

### Bacteria;“Firmicutes”;“Bacilli”;“Lactobacillales”;“Enterococcaceae”;Enterococcus,

### Bacteria;“Firmicutes”;“Clostridia”;Clostridiales;“Eubacteriaceae”;Eubacterium

Bacteria;“Firmicutes”;“Clostridia”;Clostridiales;“Eubacteriaceae”;unclassified_“Eubacteriaceae”,

### Bacteria;“Firmicutes”;“Clostridia”;Clostridiales;“Lachnospiraceae”;Anaerostipes,

### Bacteria;“Firmicutes”;“Clostridia”;Clostridiales;“Lachnospiraceae”;Dorea,

### Bacteria;“Firmicutes”;“Clostridia”;Clostridiales;“Lachnospiraceae”;Roseburia,

Bacteria;“Firmicutes”;“Clostridia”;Clostridiales;“Lachnospiraceae”;unclassified_“Lachnospiraceae”,

### Bacteria;“Firmicutes”;“Clostridia”;Clostridiales;“Peptostreptococcaceae”;Sporacetigenium,

Bacteria;“Firmicutes”;“Clostridia”;Clostridiales;“Peptostreptococcaceae”;unclassified_“Peptostreptococcaceae”,

### Bacteria;“Firmicutes”;“Clostridia”;Clostridiales;“Ruminococcaceae”;Anaerofilum,

### Bacteria;“Firmicutes”;“Clostridia”;Clostridiales;“Ruminococcaceae”;Anaerotruncus,

### Bacteria;“Firmicutes”;“Clostridia”;Clostridiales;“Ruminococcaceae”;Oscillibacter,

Bacteria;“Firmicutes”;“Clostridia”;Clostridiales;“Ruminococcaceae”;unclassified“Ruminococcaceae”,

### Bacteria;“Firmicutes”;“Clostridia”;Clostridiales;Clostridiaceae;“Clostridiaceae1”;Clostridium,

### Bacteria;“Firmicutes”;“Clostridia”;Clostridiales;Clostridiaceae;“Clostridiaceae1”;Sarcina,

Bacteria;“Firmicutes”;“Clostridia”;Clostridiales;Clostridiaceae;“Clostridiaceae1”;unclassified_“Clostridiaceae1”,

### Bacteria;“Firmicutes”;“Clostridia”;Clostridiales;IncertaeSedisXI;Anaerococcus,

### Bacteria;“Firmicutes”;“Clostridia”;Clostridiales;IncertaeSedisXI;Finegoldia,

### Bacteria;“Firmicutes”;“Clostridia”;Clostridiales;IncertaeSedisXI;Peptoniphilus,

### Bacteria;“Firmicutes”;“Clostridia”;Clostridiales;IncertaeSedisXI;unclassified_IncertaeSedisXI,

### Bacteria;“Firmicutes”;“Clostridia”;Clostridiales;IncertaeSedisXIII;Anaerovorax,

### Bacteria;“Firmicutes”;“Clostridia”;Clostridiales;IncertaeSedisXIII;Mogibacterium,

### Bacteria;“Firmicutes”;“Clostridia”;Clostridiales;IncertaeSedisXIV;Blautia,

### Bacteria;“Firmicutes”;“Clostridia”;Clostridiales;unclassified_Clostridiales,

### Bacteria;“Firmicutes”;“Clostridia”;Clostridiales;Veillonellaceae;Veillonella,

### Bacteria;“Firmicutes”;“Erysipelotrichi”;“Erysipelotrichales”;Erysipelotrichaceae;Holdemania,

### Bacteria;“Proteobacteria”;Betaproteobacteria;Burkholderiales;Alcaligenaceae;Parasutterella,

According to one embodiment of the invention the bacterial content comprises one or more of the above mentioned bacteria species.

Diseases that can be treated or prevented by the invention are for example diarrhoeas following antibiotic treatment, especially diarrhoeas caused by Clostridium difficile which can be very long lasting and difficult to treat by conventional methods and can in rare cases lead to death. Other therapeutic areas are Irritable Bowel Syndrome (IBS), Celiac Disease and Inflammatory Bowel Diseases for example Ulcerative Colitis, Crohn's Disease, Microscopic Colitis, and Pauchitis. Another treatment area is iatrogenic disturbance/dysbiosis of the intestinal microbiome, e.g. following radiation therapy, chemotherapy and in connection with transplantations. Examples of other diseases that can be treated or prevented with the invention are: neurological diseases for example Parkinson's Disease, Alzheimer's Disease, Lou Gehrig's Disease ALS-Amyotrophic Lateral Sclerosis, Multiple Sclerosis; behavioural/psychiatric disorders e.g. Autism, Asperger's Syndrome , Attention Deficit Hyperactivity Disorder (ADHD) and Depression; Rheumatologic diseases e.g. Rheumatoid arthritis; and systemic/metabolic disorders for example: Hypertension, Obesitas and type-2 Diabetes, and neoplastic/tumour diseases of the gastrointestinal tract. The invention can also be used in the treatment of Chronic Fatigue Syndrome. The invention can also be used in the treatment of dermatological diseases such as acne vulgaris.

The invention also relates to the use of such a anaerobically cultivated human microbiota for the production of a pharmaceutical treatment or prevention of disease.

The composition according to the present invention may be administrated to a human by a device selected from a naso-duodenal tube, gastroscope, colo/sigmoideoscope, and enema, or in freeze dried form in a suitable galenic preparation, e.g. gastric acid resistant capsule, nano-encapsulation or suppository.

In one embodiment the composition may also contain other microbial factors (e.g. bacteriophages) or signalling substances active against diseases.

### Forms of Preparations

The composition may be provided in the following forms of preparations:
1. Freshly thawn from frozen solution
2. Freeze-dried cultivated product
3. Fresh from the culture
4. Live micro-encapsulated culture

### Dosage

The invention is applied to the human gastro-intestinal tract at least one time per day during the course of a number of days, e.g. 1-30 days, depending on the nature, cause and severity of the problem. Per administration 1-100 ml of the product is given, preferably 20-40 ml, or the equivalent amount in freeze-dried form.

### Means of Administration

The product can preferably be administered according to the following non limiting examples: in the form of a solution through e.g. a naso-duodenal tube, gastroscope, colo/sigmoideoscope, enema or in freeze dried form in a suitable galenic preparation, e.g. a gastric acid resistant capsule, nano-encapsulated or a suppository.

### Other Ingredients (Vehicles)

Other substances and components which facilitates the administration or supports the effect of the invention may be added to the composition.

The treatment with the invention may or may not be preceded by an antibiotic treatment andor motility reducing substances.

The invention is described by the following non limiting examples.

### Example 1

Microbiota according to the present invention were obtained from a stool sample from a healthy single donor prior to 1995. Both the donor and the stool were thoroughly examined. The functional status of the donor's intestinal microbiota were found to have normal functional values of coprostanol, urobilins, mucin, fecal tryptic activity, short chain fatty acids and beta-aspartylglycine. The tests for hepatitis viruses A, B and C, cytomegalovirus, Epstein-Barr virus, human immunodeficiency virus (HIV), Calici- and Rotavirus were all negative. Furthermore, the faeces was screened for presence of Salmonella, Shigella, Campylobacter, Yersinia, Clostridium difficile and protozoan cysts, and all these investigations were negative.

A sample of faeces from the interior of the stool sample (Bristol scale type 2) was taken from the above-mentioned healthy donor and immediately suspended in 30 ml of a pre-reduced sterilized bacteriological peptone yeast based medium (Difco, USA), with addition of 1.25% (wv) freeze dried hen yolk, Fresenius-Kabi Sweden with resazurin as an anaerobic indicator. This composition was re-cultivated under nitrogen flow every second week. The bacterial content of the cultivated product were found to be >10⁹ per ml medium.

The product contained the following species:

### Bacteria;“Actinobacteria”;Actinobacteria;Coriobacteridae;Coriobacteriales;“Coriobacterineae”;Coriobacteriaceae;Collinsella,

### Bacteria;“Actinobacteria”;Actinobacteria;Coriobacteridae;Coriobacteriales;“Coriobacterineae”;Coriobacteriaceae; Eggerthella,

### Bacteria;“Actinobacteria”;Actinobacteria;Coriobacteridae;Coriobacteriales;“Coriobacterineae”;Coriobacteriaceae;Olsenella,

Bacteria;“Bacteroidetes”;“Bacteroid ia”;“Bacteroidales”;“Porphyromonadaceae”; Parabacteroides,
Bacteria;“Bacteroidetes”;“Bacteroid ia”;“Bacteroidales”;“Prevotel laceae”; Prevotella,

### Bacteria;“Bacteroidetes”;“Bacteroidia”;“Bacteroidales”;“Rikenellaceae”;Alistipes,

### Bacteria;“Bacteroidetes”;“Bacteroidia”;“Bacteroidales”; Bacteroidaceae; Bacteroides

### Bacteria;“Firmicutes”;“Bacilli”;“Lactobacillales”;“Enterococcaceae”;Enterococcus,

### Bacteria;“Firmicutes”;“Clostridia”;Clostridiales;“Eubacteriaceae”;Eubacterium

Bacteria;“Firmicutes”;“Clostridia”;Clostridiales;“Eubacteriaceae”;unclassified“Eubacteriaceae”,

### Bacteria;“Firmicutes”;“Clostridia”;Clostridiales;“Lachnospiraceae”;Anaerostipes,

### Bacteria;“Firmicutes”;“Clostridia”;Clostridiales;“Lachnospiraceae”;Dorea,

### Bacteria;“Firmicutes”;“Clostridia”;Clostridiales;“Lachnospiraceae”;Roseburia,

Bacteria;“Firmicutes”;“Clostridia”;Clostridiales;“Lachnospiraceae”;unclassified_“Lachnospiraceae”,

### Bacteria;“Firmicutes”;“Clostridia”;Clostridiales;“Peptostreptococcaceae”;Sporacetigenium,

Bacteria;“Firmicutes”;“Clostridia”;Clostridiales;“Peptostreptococcaceae”;unclassified_“Peptostreptococcaceae”,

### Bacteria;“Firmicutes”;“Clostridia”;Clostridiales;“Ruminococcaceae”;Anaerofilum,

### Bacteria;“Firmicutes”;“Clostridia”;Clostridiales;“Ruminococcaceae”;Anaerotruncus,

### Bacteria;“Firmicutes”;“Clostridia”;Clostridiales;“Ruminococcaceae”;Oscillibacter,

Bacteria;“Firmicutes”;“Clostridia”;Clostridiales;“Ruminococcaceae”;unclassified_“Ruminococcaceae”,

### Bacteria;“Firmicutes”;“Clostridia”;Clostridiales;Clostridiaceae;“Clostridiaceae1”;Clostridium,

### Bacteria;“Firmicutes”;“Clostridia”;Clostridiales;Clostridiaceae;“Clostridiaceae1”;Sarcina,

Bacteria;“Firmicutes”;“Clostridia”;Clostridiales;Clostridiaceae;“Clostridiaceae1”;unclassified_“Clostridiaceae1”,

### Bacteria;“Firmicutes”;“Clostridia”;Clostridiales;IncertaeSedisXI;Anaerococcus,

### Bacteria;“Firmicutes”;“Clostridia”;Clostridiales;IncertaeSedisXI;Finegoldia,

### Bacteria;“Firmicutes”;“Clostridia”;Clostridiales;IncertaeSedisXI;Peptoniphilus,

### Bacteria;“Firmicutes”;“Clostridia”;Clostridiales;IncertaeSedisXI;unclassified_IncertaeSedisXI,

### Bacteria;“Firmicutes”;“Clostridia”;Clostridiales;IncertaeSedisXIII;Anaerovorax,

### Bacteria;“Firmicutes”;“Clostridia”;Clostridiales;IncertaeSedisXIII;Mogibacterium,

### Bacteria;“Firmicutes”;“Clostridia”;Clostridiales;IncertaeSedisXIV;Blautia,

### Bacteria;“Firmicutes”;“Clostridia”;Clostridiales;unclassified_Clostridiales,

### Bacteria;“Firmicutes”;“Clostridia”;Clostridiales;Veillonellaceae;Veillonella,

### Bacteria;“Firmicutes”;“Erysipelotrichi”;“Erysipelotrichales”;Erysipelotrichaceae;Holdemania, and

### Bacteria;“Proteobacteria”;Betaproteobacteria;Burkholderiales;Alcaligenaceae;Parasutterella,

After addition of 10% glycerol the composition can be stored at −70° C.

### Example 2

The fresh or frozen stool sample from an individual who has not been exposed to antibiotics or antibiotic resistance genes e.g. through a bacterial infection, is inoculated under anaerobic conditions on to 30 ml peptone-yeast medium (Difco, USA), containing cholesterol of animal origin (e.g. 1.25% freeze-dried hen yolk, Fresenius-Kabi,Sweden), with resazurin as an anaerobic indicator. This composition is re-cultivated every second week. The bacterial content of the cultivated product should be >10⁶ preferably >10⁹ per ml medium. After addition of 10% glycerol the composition can be stored at −70° C.

### Example 3

A number of 32 patients, aged 27-94 years (20 females, median age 78 years and 12 males, median age 75 years) were treated with a composition according to the invention. Out of these patients, 22 had underlying chronic diseases as cancer (breast, colon, prostate) and cardiovascular or pulmonary diseases. In 11 patients cephalosporins were given, in 9 patients clindamycin were given, in 4 patients ciprofloxacin were given, in 2 patients isoxazolylpenicillin were given, another 3 patients were given some other antibiotics, and finally, 1 patient was given azitromycin when recurrent Clostridium Difficile infection (RCDI) first was diagnosed. 2 patients did not obtain specific antibiotic pretreatment before receiving the composition according to the invention.

Routinely, a volume of 30 ml of the invention according to Example 1 was given rectally by a rectally introduced catheter in Sigmoideum or by colonoscopy. Analysis of clostridium toxin was performed according to general practice at Karolinska Hospital, Huddinge, Sweden. Out of the 32 patients receiving the invention, 22 patients were cured. These 22 patients experienced a dramatic improvement in quality of life from living with diarrhoea during several months to a life with normal bowel habits within some few days. Fifteen of these patients received the invention only once, the remaining received the invention one to two more times. Four more patients experienced a considerable improvement, and in the remaining 4 patients repeated antibiotic therapies had to be given. Another 2 patients with unknown cause of the diarrhoea were also cured. The follow-up time was been 5-68 months, median 26 months. No adverse effects by the procedure were observed.

### Example 4

Ten patients with post-infectious gastroenteritis related to Giardia lamblia, were suffering from IBS like problems. Conventional IBS treatment was not successful. It was decided that these patients should receive the composition according to the invention. One dose of 30 ml was administrated over three consecutive days, using a gastro-jejunal tube. Surprisingly, all patients recovered shortly after the administration of the invention, and remained without symptoms for a long period of time.

1-24. (canceled)

25. A micro-ecological system of strictly anaerobically re-cultivated human. intestinal microbiota obtained by anaerobic cultivation of a stool sample in a cultivation medium that promotes the proliferation of the anaerobic bacteria of at least three of the following four Phyla: Bacterioidetes, Firmicutes, Proteobacteria and Actinobacteria.

26. An anaerobic micro-ecological system according to claim 25 comprising an micro-ecological system obtained from a sample from a single donor.

27. A composition comprising the anaerobic micro-ecological system according to claim 25 for the treatment or prophylaxis of diseases.

28. The composition according to claim 27 with absence of extended spectrum- and metallo-beta-lactamases, metabolites of pharmaceutical substances, or xenobiotics.

29. The composition according to claim 27, characterized in that it is free from the following microbes: hepatitis viruses A, B and C, cytomegalo virus, Epstein-Barr virus, human immunodeficiency virus (HIV), Calici- and Rotavirus, Salmonella, Shigella, Campylobacter, Yersinia, and protozoan cysts.

30. The composition according to claim 27, characterized in that the following bacterial, species together comprise more than 1% of the total bacterial content of the invention: Bacteria;“Actinobacteria”; Actinobacteria; Coriobacteridae; Coriobacteriales;“Coriobacterineae”; Coriobacteriaceae; Collinsella,
Bacteria;“Actinobacteria”;Actinobacteria;Coriobacteridae;Coriobacteriales;“Coriobacterineae”; Coriobacteriaceae;Eggerthella,
Bacteria;“Actinobacteria”;Actinobacteria;Coriobacteridae;Coriobacteriales;“Coriobacterineae”; Coriobacteriaceae;Olsenelia,
Bacteria;“Bacteroidetes”;“Bacteroidia”;“Bacteroidales”;“Porphyromonadaceae”;Parabacteroides,
Bacteria;“Bacteroidetes”;“Bacteroidia”;“Bacteroidales”;“Prevotellaceae”;Prevotella,
Bacteria;“Bacteroidetes”;“Bacteroidia”;“Bacteroidales”;“Rikenellaceae”;Alistipes,
Bacteria;“Bacteroidetes”;“Bacteroidia”;“Bacteroidales”;Bacteroidaceae;Bacteroides,
Bacteria;“Firmicutes”;“Bacilli”;“Lactobacillales”;“Enterococcaceae”;Enterococcus,
Bacteria;“Firmicutes”;“Clostridia”;Clostridiales;“Eubacteriaceae”;Eubacterium,
Bacteria;“Firmicutes”;“Clostridia”;Clostridiales;“Eubacteriaceae”;unclassified_“Eubacteriaceae”,
Bacteria;“Firmicutes”;“Clostridia”;Clostridiales;“Lachriospiraceae”;Anaerostipes,
Bacteria;“Firmicutes”;“Clostridia”;Clostridiales;“Lachnospiraceae”;Dorea
Bacteria;“Firmicutes”;“Clostridia”;Clostridiales;“Lachnospiraceae”;Roseburia,
Bacteria;“Firmicutes”;“Clostridia”;Clostridiales;“Lachnospiraceae”;
unclassified_“Lachnospiraceae”,
Bacteria;“Firmicutes”;“Clostridia”;“Clostridiales”;“Peptostreptococcaceae”;Sporaretigenium,
Bacteria;“Firmicutes”;“Clostridia”;“Clostridiales”;“Peptostreptococcaceae”;
unclassified_“Peptostreptococcaceae”,
Bacteria;“Firmicutes”;“Clostridia”;“Clostridiales”;“Ruminococcaceae”;Anaerofilum,
Bacteria;“Firmicutes”;“Clostridia”;“Clostridiales”;“Ruminococcaceae”;Anaerotruncus,
Bacteria;“Firmicutes”;“Clostridia”;“Clostridiales”;“Ruminococcaceae”;Oscillibacter,
Bacteria;“Firmicutes”;“Clostridia”;“Clostridiales”;“Ruminococcaceae”;
unclassified_“Ruminococcaceae”,
Bacteria;“Firmicutes”;“Clostridia”;Clostridiales;Clostridiaceae;“Clostridiaceae1”;Clostridium,
Bacteria;“Firmicutes”;“Clostridia”;Clostridiales;Clostridiaceae;“Clostridiaceae1”;Sarcina,
Bacteria;“Firmicutes”;“Clostridia”;Clostridiales;Clostridiaceae;“Clostridiaceae1”;
unclassified_“Clostridiaceae1”,
Bacteria;“Firmicutes”;“Clostridia”;Clostridiales;IncertaeSedisXI;Anaerococcus,
Bacteria;“Firmicutes”;“Clostridia”;Clostridiales;IncertaeSedisXI;Finegoldia,
Bacteria;“Firmicutes”;“Clostridia”;Clostridiales;IncertaeSedisXI;Peptoniphilus,
Bacteria;“Firmicutes”;“Clostridia”;Clostridiales;IncertaeSedisXI;unclassified_IncertaeSedisXI,
Bacteria;“Firmicutes”;“Clostridia”;Clostridiales;IncertaeSedisXIII;Anaerovorax,
Bacteria;“Firmicutes”;“Clostridia”;Clostridiales;IncertaeSedisXIII;Mogibacterium,
Bacteria;“Firmicutes”;“Clostridia”;Clostridiales;IncertaeSedisXIV;Blautia,
Bacteria;“Firmicutes”;“Clostridia”;Clostridiales;unclassified_Clostridiales,
Bacteria;“Firmicutes”;“Clostridia”;Clostridiales;Veillonellaceae;Veillonella,
Bacteria;“Firmicutes”;“Erysipelotrichi”;“Erysipelotrichales”;Erysipelotrichaceae;Holdemania, and
Bacteria;“Proteobacteria”;Betaproteobacteria;Burkholderiales;Alcaligenaceae;Parasutterella

31. The composition according to claim 27, characterized in that the bacterial content of the composition comprises one or more of
Bacteria;“Actinobacteria”;Actinobacteria;Coriobacteridae;Coriobacteriales;“Coriobacterineae”; Coriobacteriacea;Collinsella,
Bacteria;“Actinobacteria”;Actinobacteria;Coriobacteridae;Coriobacteriales;“Coriobacterineae”; Coriobacteriaceae; Eggerthella,
Bacteria;“Actinobacteria”;Actinobacteria;Coriobacteridae;Coriobacteriales;“Coriobacterineae”; Coriobacteriaceae;Olsenella,
Bacteria;“Bacteroidetes”;“Bacteroidia”;“Bacteroidales”;“Porphyromanadaceae”;Parabacteroides,
Bacteria;“Bacteroidetes”;Bacteroidia”;“Bacteroidales”;“Prevotellaceae”;Prevotella,
Bacteria;“Bacteroidetes”;Bacteroidia”;“Bacteroidales”;“Rikenellaceae”;Alistipes,
Bacteria;“Bacteroidetes”;Bacteroidia”;“Bacteroidales”;Bacteroidaceae;Bacteroides,
Bacteria;“Firmicutes”;“Bacilli”;“Lactobacillales”;“Enterococcaceae”;Enterococcus,
Bacteria;“Firmicutes”;“Clostridia”;Clostridiales;“Eubacteriaceae”;Eubacterium,
Bacteria;“Firmicutes”;“Clostridia”;Clostridiales;“Eubacteriaceae”;unclassified_“Eubacteriaceae”,
Bacteria;“Firmicutes”;“Clostridia”;Clostridiales;“Lachnospiraceae”;Anaerostipes,
Bacteria;“Firmicutes”;“Clostridia”;Clostridiales;“Lachnospiraceae”;Dorea,
Bacteria;“Firmicutes”;“Clostridia”;Clostridiales;“Lachnospiraceae”;Roseburia,
Bacteria;“Firmicutes”;“Clostridia”;Clostridiales;“Lachnospiraceae”;
unclassified_“Lachnospiraceae”,
Bacteria;“Firmicutes”;“Clostridia”;Clostridiales;“Peptostreptococcaceae”;Sporacetigenium,
Bacteria;“Firmicutes”;“Clostridia”;Clostridiales;“Peptostreptococcaceae”;
unclassified_;“Peptostreptococcaceae”;
Bacteria;“Firmicutes”;“Clostridia”;Clostridiales;“Ruminococcaceae”;Anaerofilum,
Bacteria;“Firmicutes”;“Clostridia”;Clostridiales;“Ruminococcaceae”;Anaerotruncus,
Bacteria;“Firmicutes”;“Clostridia”;Clostridiales;“Ruminococcaceae”;Oscillibacter,
Bacteria;“Firmicutes”;“Clostridia”;Clostridiales;“Ruminococcaceae”;
unclassified_“Ruminococcaceae”,
Bacteria;“Firmicutes”;“Clostridia”;Clostridiales;Clostridiaceae;“Clostridiaceae1”;Clostridium,
Bacteria;“Firmicutes”;“Clostridia”;Clostridiales;Clostridiaceae;“Clostridiaceae1”;Sarcina,
Bacteria;“Firmicutes”;“Clostridia”;Clostridiales;Clostridiaceae;“Clostridiaceae1”;
unclassified_“Clostridiaceae1”,
Bacteria;“Firmicutes”;“Clostridia”;Clostridiales;IncertaeSedisXI;Anaercoccus,
Bacteria;“Firmicutes”;“Clostridia”;Clostridiales;IncertaeSedisXI;Finegoldia,
Bacteria;“Firmicutes”;“Clostridia”;Clostridiales;IncertaeSedisXI;Peptoniphilus,
Bacteria;“Firmicutes”;“Clostridia”;Clostridiales;IncertaeSedisXI;unclassified_IncertaeSedisXI,
Bacteria;“Firmicutes”;“Clostridia”;Clostridiales;IncertaeSedisXIII;Anaerovorax,
Bacteria;“Firmicutes”;“Clostridia”;Clostridiales;IncertaeSedisXII;Mogibacterium,
Bacteria;“Firmicutes”;“Clostridia”;Clostridiales;IncertaeSedisXIV;Blautia,
Bacteria;“Firmicutes”;“Clostridia”;Clostridiales;unclassified_Clostridiales,
Bacteria;“Firmicutes”;“Clostridia”;Clostridiales;Veillonellaceae;Veillonella,
Bacteria;“Firmicutes”;“Erysipelotrichi”;“Erysipelotrichales”;Erysipelotrichaceae;Holdemania, and
Bactetia;“Proteobacteria”;Betaproteobacteria;Burkholderiales;Alcaligenaceae;Parasutterella.

32. The composition according to claim 27 for use as a pharmaceutical.

33. The composition according to claim 27 for use in the treatment of diarrhea, such as Irritable Bowel Syndrome (IBS), or antibiotic associated diarrhea(AAD), especially related to Clostridium difficile, Clostridium difficile associated diarrhoea (CDAD) and recurrent Clostridium difficile infections (RCDI).

34. The composition according to claim 27 for use in administration to a human by a device selected from a naso-duodenal tube, gastroscope, colo/sigmoideoscope, and enema, or in freeze dried form in a suitable galenic preparation, e.g. gastric acid resistant capsule, nano-encapsulated or suppository.

35. A method for preparing a composition comprising an anaerobic microecological system comprising anaerobic human intestinal microbiota, the method characterized by anaerobic cultivation of a stool sample in a cultivation medium that promotes the proliferation of the anaerobic bacteria of at least three of the following our Phyla: Bacterioidetes, Firmicutes, Proteobacteria and Actinobacteria.

36. A method according to claim 35, characterized in that aerobic bacteria are suppressed during cultivation.
